(19) United States
(12) Patent Application Publication
(10) Pub. No.: US 2022/0193317 A1
(43) Pub. Date: Jun. 23, 2022

(54) SILICONE RUBBER HEATER FOR PERITONEAL DIALYSIS

(71) Applicant: Bellco SRL, Mirandola (IT)

(72) Inventor: Antonio Guiseppe Marra, Bologna (IT)

(73) Assignee: Bellco SRL, Mirandola (IT)

(21) Appl. No.: 17/551,446

(22) Filed: Dec. 15, 2021

(30) Foreign Application Priority Data
Dec. 22, 2020 (EP) 20216613.8

(51) Int. Cl. A61M 1/16; H05B 3/34; H05B 3/14

(52) U.S. Cl. CPC A61M 1/1664; A61M 1/1668; H05B 2203/01; H05B 3/148; A61M 2205/36; H05B 3/34

(57) ABSTRACT

The disclosure relates to a heater made from a layer of silicone rubber or similar material having one or more embedded heating elements and a conductive plate such as an aluminum plate positioned on a side of the layer of silicone rubber. The heater can be used to heat fluids necessary to generate a peritoneal dialysis fluid or heat peritoneal dialysis fluid prior to infusion into a patient. Systems that have one or more of the heaters of the invention to generate the peritoneal dialysis fluid and to heat the generated peritoneal dialysis fluid are provided.

### FIELD

The disclosure relates to a heater made from a layer of silicone rubber or similar material having one or more embedded heating elements and a conductive plate such as an aluminum plate positioned on a side of the layer of silicone rubber. The heater having a layer of silicone rubber with a corresponding conductive plate can be shaped into many different sizes and shapes to uniformly heat a fluid receptacle of any size or shape in any heating compartment wherein the compartment also can have any size of shape. The heater can be used in any application where a fluid must be heated uniformly in a receptacle of any size of placement. The heater can be used to heat fluids necessary to generate a peritoneal dialysis fluid or heat peritoneal dialysis fluid prior to infusion into a patient. Systems that have one or more of the heaters of the invention to generate the peritoneal dialysis fluid and to heat the generated peritoneal dialysis fluid are provided.

### BACKGROUND

Heaters often fail to uniformly or accurately heat fluids to a specific temperature. The problem can be aggravated where the heater is required to heat fluids or water in fluid receptacles of different sizes and shapes. Heaters are often made from inflexible materials such as ceramic and cannot be easily engineered into tight spaces, unusually shaped designs, or specific configurations to uniformly heat fluid receptacles or containers of various shapes and sizes. Yet accurate and precise heating control is often required in many applications where heat is delivered to fluid in order to reach or attain a specific fluid temperature. The known heaters and systems often fail to heat fluids to a specific temperature due to the creation of hot or cold spots on the surface of the heater that translate into non-uniform heating of a fluid receptacle or container. The known heaters often fail to provide uniform heating because they contain hot or cold spots. Because of the high heating power density of certain heaters, such as ceramic heaters, the fluids in the bags or containers do not heat uniformly. This can cause problems in applications where a specific temperature is required to dissolve or mix solutions or solutes such as during peritoneal dialysis. Non-uniform heating of the fluid also introduces errors into temperature measurements, as the temperature measured becomes dependent upon the location of the sensor in the container or bag. This can pose problems for creating a medical solution that requires precise and accurate temperature control to dissolve certain constituents at a particular fluid temperature.

The known heaters also sometimes fail to uniformly heat peritoneal dialysis fluid that is delivered to patients. Notably, peritoneal dialysis must be heated to body temperature prior to infusion for the health, safety, and comfort of the patient. Existing systems such as cyclers having a heater cannot heat fluid receptacles or containers of various shapes or sizes. The existing systems often are configured as a flat plate onto which a receptacle must rest. This can cause uneven heating and create a burn hazard. Known devices often fail to account for home use and cannot be engineered to size, shape, and geometry constraints.

Hence, there is a need for systems and methods that can heat fluid in a bag or container accurately, precisely, or uniformly. The need includes systems and methods that use a lower heating power density heater to more uniformly heat the fluid, improving temperature measurement accuracy and/or precision. The need extends to a heater that can be designed and fabricated into various shapes, sizes, and geometries to accommodate fluid bags or containers of various shapes, sizes, and geometries. The need extends to heaters that can fit inside various receiving compartments or spaces insides devices. The need extends further to heaters that can be engineered into tight spaces, unusually shaped designs, or specific configurations. The need includes accurate and precise heating control so that a fluid container being heated by the heater uniformly reaches or attains a specific fluid temperature quickly and accurately. The need includes further systems that can heat fluids to a specific temperature without overheating or underheating specific spots on a fluid receptacle or container.

### SUMMARY OF THE INVENTION

The problem to be solved by the present invention is accurate and uniform heating of fluids used in generating fluid to dissolve concentrates for use in peritoneal dialysis fluid and/or in heating a peritoneal dialysis fluid to a suitable temperature for treating a patient with peritoneal dialysis fluid. The solution can use a heater that has a low heating power density made from a flexible material layer such as silicone rubber having heating elements embedded therein, that is affixed or adhered to a conductive plate such as an aluminum plate to uniformly heat a fluid in a bag.

The first aspect of the invention relates to a heater. In any embodiment, the heater can include a first layer of silicone rubber; the first layer of silicone rubber having one or more heating elements embedded in the silicone rubber; and a conductive plate; the conductive plate positioned on a first side of the first layer of silicone rubber.

In any embodiment, conductive plate can be selected from any one of an aluminum plate, silver plate, copper plate, gold plate, silicon carbide plate, tungsten plate, graphite plate, zinc plate, and any combination thereof.

In any embodiment, the conductive plate can be selected from a thermally conductive metal, thermally conductive metal alloy, or thermally conductive composite.

In any embodiment, the one or more heating elements can be uniformly distributed through the first layer of silicone rubber.

In any embodiment, the first layer of silicone rubber and the conductive plate can have a concave shape.

In any embodiment, the convex shape can be substantially round.

In any embodiment, an area of the conductive plate can be substantially similar to an area of the first side of the first layer of silicone rubber.

In any embodiment, the silicone rubber can be selected from any one or more of a polysiloxane or polydimethylsiloxane.

The features disclosed as being part of the first aspect of the invention can be in the first aspect of the invention, either alone or in combination, or follow any arrangement or permutation of any one or more of the described elements. Similarly, any features disclosed as being part of the first aspect of the invention can be in a second, third, fourth, fifth, or sixth aspect of the invention described below, either alone or in combination, or follow any arrangement or permutation of any one or more of the described elements.

The second aspect of the invention relates to a system. In any embodiment, the system can include a peritoneal dialysis cycler; and the heater of the first aspect of the invention.

In any embodiment, the peritoneal dialysis cycler can include a substantially flat surface; the first layer of silicone rubber and the conductive plate positioned on the substantially flat surface.

In any embodiment, the peritoneal dialysis cycler can include a receiving compartment for a fluid bag; the first layer of silicone rubber and the conductive plate lining an inner surface of the receiving compartment.

In any embodiment, the receiving compartment can be substantially rectangular.

In any embodiment, the receiving compartment can be substantially round.

The features disclosed as being part of the second aspect of the invention can be in the second aspect of the invention, either alone or in combination, or follow any arrangement or permutation of any one or more of the described elements. Similarly, any features disclosed as being part of the second aspect of the invention can be in the first, third, fourth, fifth, or sixth aspects of the invention, either alone or in combination, or follow any arrangement or permutation of any one or more of the described elements.

The third aspect of the invention relates to a system. In any embodiment, the system can include a peritoneal dialysis fluid generation module; and the heater of the first aspect of the invention.

In any embodiment, the peritoneal fluid generation module can include a substantially flat surface; the first layer of silicone rubber and the conductive plate positioned on the substantially flat surface.

In any embodiment, the peritoneal dialysis fluid generation module can include a receiving compartment for a fluid bag; the first layer of silicone rubber and the conductive plate lining an inner surface of the receiving compartment.

In any embodiment, the receiving compartment can be substantially rectangular.

In any embodiment, the receiving compartment can be substantially round.

The features disclosed as being part of the third aspect of the invention can be in the third aspect of the invention, either alone or in combination, or follow any arrangement or permutation of any one or more of the described elements. Similarly, any features disclosed as being part of the third aspect of the invention can be in the first, second, fourth, fifth, or sixth aspects of the invention, either alone or in combination, or follow any arrangement or permutation of any one or more of the described elements.

The fourth aspect of the invention relates to a method. In any embodiment, the method can include the steps: of heating a fluid bag with the heater of the first aspect of the invention; the dialysis fluid bag containing a peritoneal dialysis fluid; and infusing the peritoneal dialysis fluid from the dialysis fluid bag into a patient.

In any embodiment, the step of heating the dialysis fluid bag can include positioning the fluid bag into a receiving compartment of a peritoneal dialysis cycler.

The features disclosed as being part of the fourth aspect of the invention can be in the fourth aspect of the invention, either alone or in combination, or follow any arrangement or permutation of any one or more of the described elements. Similarly, any features disclosed as being part of the fourth aspect of the invention can be in the first, second, third, fifth, or sixth aspects of the invention, either alone or in combination, or follow any arrangement or permutation of any one or more of the described elements.

The fifth aspect of the invention relates to a method. In any embodiment, the method can include the steps of heating purified water in a fluid bag with the heater of the first aspect of the invention; and generating a peritoneal dialysis fluid using the purified water.

In any embodiment, the step of heating the purified water bag can include positioning the fluid bag into a receiving compartment of a peritoneal dialysis fluid generation module.

In any embodiment, the method can include the steps of pumping the purified water into a concentrate source to generate a concentrate; the concentrate source initially containing a solid material.

In any embodiment, the method can include the steps of the steps of pumping the purified water into at least two concentrate sources to generate at least two concentrates; the at least two concentrate sources each initially containing a solid material.

The features disclosed as being part of the fifth aspect of the invention can be in the fifth aspect of the invention, either alone or in combination, or follow any arrangement or permutation of any one or more of the described elements. Similarly, any features disclosed as being part of the fifth aspect of the invention can be in the first, second, third, fourth, or sixth aspects of the invention, either alone or in combination, or follow any arrangement or permutation of any one or more of the described elements.

The sixth aspect of the invention relates to a system. In any embodiment, the system can include a peritoneal dialysis cycler wherein the peritoneal dialysis cycler includes the heater of the first aspect of the invention and a peritoneal dialysis fluid generation module wherein the peritoneal dialysis fluid generation module also includes the heater of the first aspect of the invention.

The features disclosed as being part of the sixth aspect of the invention can be in the sixth aspect of the invention, either alone or in combination, or follow any arrangement or permutation of any one or more of the described elements. Similarly, any features disclosed as being part of the sixth aspect of the invention can be in the first, second, third, fourth, or fifth aspects of the invention, either alone or in combination, or follow any arrangement or permutation of any one or more of the described elements.

### BRIEF DESCRIPTION OF THE DRAWINGS

FIGS. 1A-D illustrate a peritoneal dialysis system having a substantially flat silicone rubber heater.

FIGS. 2A-D illustrate a peritoneal dialysis system having a substantially rectangular receiving compartment for heating a fluid bag.

FIG. 3 illustrates a peritoneal dialysis system having a substantially rectangular receiving compartment with a lid for heating a fluid bag.

FIGS. 4A-D illustrate a peritoneal dialysis system having a triangular receiving compartment for heating a fluid bag.

FIG. 5 illustrates a peritoneal dialysis system having a triangular receiving compartment with a lid for heating a fluid bag.

FIGS. 6A-D illustrate a peritoneal dialysis system having a substantially round receiving compartment for heating a fluid bag.

FIG. 7 illustrates a peritoneal dialysis system having a substantially round receiving compartment with a lid for heating a fluid bag.

FIGS. 8A-D illustrate a peritoneal dialysis system having a hexagonal receiving compartment for heating a fluid bag.

FIG. 9 illustrates a peritoneal dialysis system having a hexagonal receiving compartment with a lid for heating a fluid bag.

FIG. 10 illustrates a peritoneal dialysis system including a cycler and generation module each using a silicone rubber heater.

FIG. 11 is a flow diagram of a peritoneal dialysis generation module.

FIG. 12 is an experimental setup used to test a silicone rubber heater.

FIGS. 13A-C are graphs of temperature vs. time for heating fluid in a bag with a silicone rubber heater.

FIGS. 14A-C are images from an experiment to determine the uniformity of temperature using a silicone rubber heater.

### DETAILED DESCRIPTION

Unless defined otherwise, all technical and scientific terms used have the same meaning as commonly understood by one of ordinary skill in the art.

The articles “a” and “an” are used to refer to one to over one (i.e., to at least one) of the grammatical object of the article. For example, “an element” means one element or over one element.

An “aluminum plate” can be a piece of aluminum metal including aluminum alloys containing any percentage of other suitable metal or material known to those of skill in the art for appropriate thermal conductivity. The piece can be of any planar shape, size, or thickness.

The term “comprising” includes, but is not limited to, whatever follows the word “comprising.” Use of the term indicates the listed elements are required or mandatory but that other elements are optional and may be present.

The term “consisting of” includes and is limited to whatever follows the phrase “consisting of.” The phrase indicates the limited elements are required or mandatory and that no other elements may be present.

The term “consisting essentially of” includes whatever follows the term “consisting essentially of” and additional elements, structures, acts, or features that do not affect the basic operation of the apparatus, structure or method described.

The term “concave” refers to a shape of a material that is curved inwardly.

A “concentrate” refers to a solution of solutes in water; the solution having a higher concentration than intended for use in treatment.

A “concentrate source” refers to a container from which a concentrate can be obtained.

A “copper plate” can be a piece of copper metal including copper alloys containing any percentage of other suitable metal or material known to those of skill in the art for appropriate thermal conductivity. The piece can be of any planar shape, size, or thickness.

The term “embedded” refers to a first component being fixed within completely or partially a second component or material.

To “generate” a fluid refers to a process of creating the fluid from constituent parts.

A “gold plate” can be a piece of gold metal including gold alloys containing any percentage of other suitable metal or material known to those of skill in the art for appropriate thermal conductivity. The piece can be of any planar shape, size, or thickness.

A “graphite plate” can be a piece of graphite including graphite composites containing any percentage of other suitable metal or material known to those of skill in the art for appropriate thermal conductivity. The piece can be of any planar shape, size, or thickness.

A “heater” is a component that is used to raise the temperature of container or substance.

The terms “heating” or to “heat” refer to a process of raising a temperature of a substance or container.

A “heating element” is an electrical component that increases in temperature as an electrical current flows through the component.

To “infuse” a fluid refers to the process of flowing a fluid into a cavity.

An “inner surface” of a component refers to a wall of the component that is inside of the component or system.

The term “initially” refers to a state of a component or system prior to a process.

A “layer of silicone rubber” refers to an elastomeric material composed of a silicone-based polymer of any suitable type known to those of skill in the art.

The term “lining” or to “line” refers to a first material or component covering a surface of a second material or component.

The term “on top” refers to the relative position of two components wherein a first component is positioned above a second component when arranged for normal use. The first component is on top of the second component.

A “peritoneal dialysis cycler” is a component or set of components for movement of fluid into and out of the peritoneal cavity of a patient.

“Peritoneal dialysis fluid” is a dialysis solution to be used in peritoneal dialysis having specified parameters for purity and sterility. Peritoneal dialysis fluid is generally not the same as dialysate used in hemodialysis.

A “fluid bag” is a bag or container that contains any fluid or aqueous solution. One type of “fluid bag” is a “peritoneal dialysis fluid bag” that can contain either peritoneal dialysis fluid or any fluid used in generating peritoneal dialysis fluid.

A “peritoneal dialysis fluid generation module” is a component or set of components for generating peritoneal dialysis fluid from one or more solid substances, concentrates, or solutions containing components of peritoneal dialysis fluid.

The term “polydimethylsiloxane” also known as dimethylpolysiloxane or dimethicone, refers to a group of polymeric organosilicon compounds that are commonly referred to as silicones.

The term “polysiloxane” refers to any polymer having an inorganic backbone of —(Si—O)— repeat units.

The term “position” or “positioned” refers to a physical location of a component or system.

The terms “pumping” or to “pump” refer to moving a fluid or gas using suction or pressure.

The term “purified water” refers to water that has been treated to remove chemical and/or biological contaminants.

A “receiving compartment” can be a compartment, section, or chamber within a larger system into which a component can be positioned.

The term “silicone rubber” refers to any elastomer containing silicon together with carbon, hydrogen, and oxygen. Silicone rubbers include any type, form, or composition of polysiloxanes or polydimethylsiloxanes. The term can also refer to any polymer having a Si—O—Si backbone. Any grade or form of silicone rubber is contemplated that is suitable for the intended use.

A “silicone carbide plate” can be a piece of silicone carbide including silicone carbide composites containing any percentage of other suitable metal or material known to those of skill in the art for appropriate thermal conductivity. The piece can be of any planar shape, size, or thickness.

The terms “solid” or “solid material” refer to a material in the solid phase of matter, and can include crystalline, powdered, or any other form of solid material.

The term “substantially flat” refers generally to the shape of a component that is smooth and even throughout. However, a “substantially flat” component can include slight variations in elevations or include small protuberances.

The term “substantially rectangular” refers generally to a three-dimensional shape of a component that has six quadrilateral faces, with opposite faces having the same dimensions. However, the actual contours of a “substantially rectangular” component can vary slightly from a perfect rectangular prism.

The term “substantially round” refers generally to the shape of a sphere or portion of a sphere. However, the actual contours of a “substantially round” component can vary slightly from a perfect spheroid.

The term “substantially similar to an area” refers to a first component having a surface area that generally has the same are as a second referenced component.

The term “thermally conductive metal” refers to any metal capable of conducting thermal heat such as copper, aluminum, brass, steel, bronze, and other similar materials known to those of skill in the art.

The term “thermally conductive metal alloy” refers to any metal combination or metal alloy that contains any percent combination of metals capable of conducting thermal heat such as copper, aluminum, brass, steel, bronze, and other similar materials known to those of skill in the art. The metal alloy can contain any suitable type of constituent such as carbon, silica, chemical additives, or elements such as lithium, sodium, or calcium. The list is non-exhaustive and includes any suitable alloy known to those of skill in the art that is a suitable heat conductor.

The term “thermally conductive composite” refers to any non-metal base material that can be used to conduct heat. For example, fibrous carbon material can be compounded in a metal matrix powder of aluminum or the like to fabricate a thermally conductive composite material. In other examples, the composite can be a conductive molded composition made from a polymer base matrix and the like. Other suitable composites known to those of skill in the art are contemplated.

A “tungsten plate” can be a piece of tungsten including tungsten composites containing any percentage of other suitable metal or material known to those of skill in the art for appropriate thermal conductivity. The piece can be of any planar shape, size, or thickness.

The term “uniformly distributed” refers to an arrangement of components embedded in a material; the arrangement substantially the same distance of the components distributed throughout the material. The arrangement can be described by any suitable configuration, geometry, or density of the component being distributed.

A “zinc plate” can be a piece of zinc including zinc composites containing any percentage of other suitable metal or material known to those of skill in the art for appropriate thermal conductivity. The piece can be of any planar shape, size, or thickness.

### Heater

The heater and related systems and methods can accurately and/or precisely heat water or fluid to a specific temperature. The heater can deliver uniform heating to accurately and/or precisely heat water or other fluids in a bag, container, or fluid receptacle made of various shapes, sizes, and materials. Specifically, the heater can be fashioned into any one or more combination of shapes and sizes that can be matched or lined to a standard device or customized to a particular configuration or design. The heaters can be used in any industrial, medical, or manufacturing process requiring precision and/or accurate heating of water or fluids. For example, the heaters can be used to heat fluid and water for chemical and plating methods, gas generation, solvent and deionized water methods, and medical applications.

In one embodiment, the heaters heat water and other fluids used to generate peritoneal dialysis. The heaters can be shaped and sized to fit inside or on top of peritoneal dialysis devices intended for home use. Often, home-use peritoneal dialysis devices must be compact or have a specific shape, geometry, or size. As such, the heaters can be fashioned to line a recessed compartment, cavity, niche, crevice, or surface of the device. The heaters can then uniformly heat water or fluid contained inside a bag shaped or sized to fit inside the heating compartment or on top of the heating surface. The uniformly heated fluid or water can then be used to dissolve and/or mix various constituents at a precise and/or accurate temperature that may be different for each constituent. The heaters of the present invention can also be used to heat any medical fluid, such as a peritoneal dialysis fluid, to a specific temperature, such as body temperature, prior to infusion into a patient. One or more heaters of the invention can be combined into a system performing one or more heating at various stages of fluid generation and therapy.

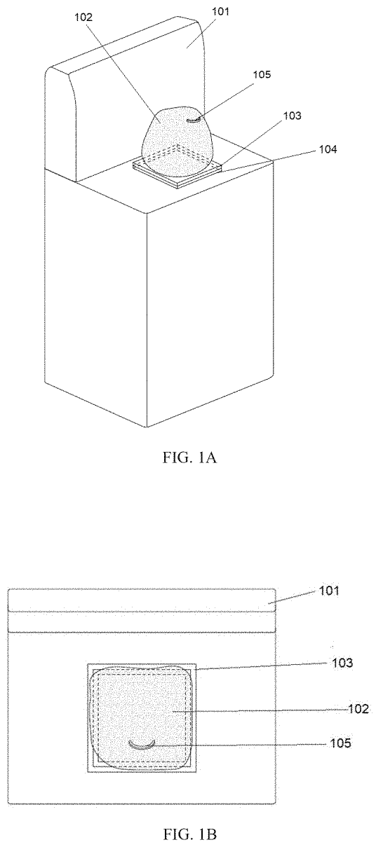

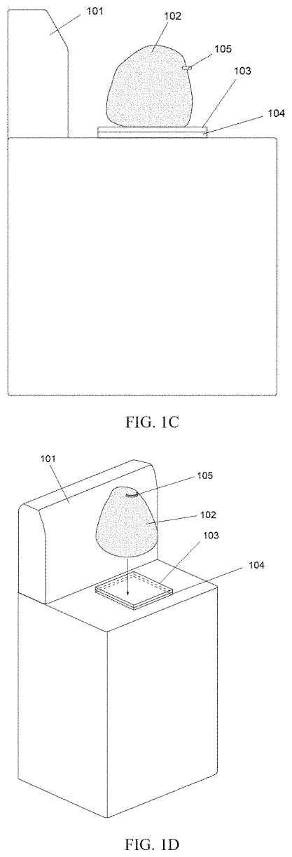

FIGS. 1A-D illustrate a peritoneal dialysis system 101 using a heater. Although a peritoneal dialysis system is provided, any industrial, medical, or manufacturing process requiring precision and/or accurate heating of water or fluids is contemplated. FIG. 1A is a perspective view of the peritoneal dialysis system 101, FIG. 1B is a top view of the peritoneal dialysis system 101, FIG. 1C is a side view of peritoneal dialysis system 101, and FIG. 1D shows a fluid bag 102 being placed onto a heater fabricated from a layer of a flexible thermally conductive material such as silicone rubber. In one embodiment, silicone rubber is contemplated and is described herein as a layer of silicone rubber 104 with embedded heating elements. The flexible conductive material can be any suitable material known to one of skill in the art having the desired properties and features capable of being embedded with heating elements. The thermally conductive silicone rubber compositions can be any thermally conductive silicones having suitable temperature and manufacturing properties. One non-limiting group of thermally conductive silicone rubber compositions include those containing a polymer base matrix, organopolysiloxanes with vinyl groups or silica groups, organohydrogenpolysiloxanes, tackifiers selected from thermally conductive fillers, aminosilanes, epoxy silanes, and alkyl titanates. One of ordinary skill in the art will understand that any suitable silicone rubber or other flexible material having any one or more appropriate features relating to tensile strength, temperature resistance, molding, heat dissipation, melting, adhesion, and/or conductive properties can be used.

In the described embodiment, the layer of flexible, thermally conductive material such as a silicone rubber 104 is fixed or adhered to a conductive plate 103 positioned on one side of the layer of silicone rubber 104. As illustrated in FIG. 1A, the heater can be positioned on a topside of a substantially flat surface of the peritoneal dialysis system 101. As described herein, any industrial, medical, or manufacturing process requiring precision and/or accurate heating of water or fluids is contemplated. The heater can be electrically integrated into the peritoneal dialysis system 101 using any appropriate connection to deliver power to the heater. The heater can include a first layer of silicone rubber 104 and a conductive plate 103 positioned on one side of the layer of silicone rubber 104 such as a topside relative to the peritoneal dialysis system 101. The layer of silicone rubber 104 can have heating elements (not shown) embedded within the silicone rubber 104. In certain embodiments, the heating elements can be embedded completely inside, partially inside, or appurtenant to the silicone rubber 104. The heating elements can be distributed in any suitable geometry or density to generate a desired watt density. Uniform or even distances between one or more heating element can encourage uniform heat distribution from the layer of silicone rubber 104 to the conductive plate 103. The one or more heating elements can be distributed through the silicone rubber 104 to provide substantially uniform radiant heating across the layer of silicone rubber 104. One of ordinary skill can use any appropriate component or method necessary to deliver power or energy to the heating elements such as a wired, wireless, direct, magnetic, inductive, or any other connection or other suitable power delivery means. The heating elements can be made from nichrome, kanthal (FeCrAl) wire, cupronickel (CuNi), or etched foil, and those particularly having a low power density. Alternatively, any heating element can be arranged in a suitable density of an amount of wattage per square inch of surface area. For example, a heating element with 10 square inches of surface area, rated for 1,500 watts, can conduct 150 watts per square inch when in use. Similarly, the same 1,500 watt element can be uniformly distributed for 7.5 square inches of surface area to produce a higher density element, conducting 200 watts per square inch. One of ordinary skill will understand that various combinations, densities, and arrangements can be used to produce a desired watt density.

The conductive plate 103 can rest on a topside of the silicone rubber 104 to encourage uniform distribution of heat radiating from the first layer of silicone rubber 104 containing the embedded heating elements. The conductive plate 103 can be affixed or adhered to the silicone rubber 104 using glue, mechanical fixation, or any other suitable process or means. In combination with the heating elements uniformly embedded inside the layer of silicone rubber 104, the conductive plate 103 can distribute heat in a uniform manner to an item to be heated. In one configuration, a fluid bag 102 can be positioned between the conductive plate 103 the silicone rubber 104. The fluid bag 102 can contain water, peritoneal dialysis fluid, fluid concentrates, combinations thereof, or any other aqueous solution. The fluid bag 102 can be any container constructed from plastic, thermoplastic, fabric, or other suitable material that can be heated at the temperatures required for a particular application. For example, the fluid bag 102 can be constructed from materials including, but not limited to, polyethylene (PE), low density polyethylene (LDPE), linear low-density polyethylene (LLDPE), polypropylene (PP), ethyl vinyl acetate (EVA), polymers, copolymers, block-copolymers, the like, and blends thereof. One of ordinary skill can make a blend of materials to produce a bag having desirable degrees of moldability, tensile strength, pliability, clarity, heat-resistance, heat-transmission, and the like.

The conductive plate 103 can be made from any thermally conductive material. For example, the thermally conductive material can be selected from aluminum, silver, copper, gold, silicone carbide, tungsten, graphite, zinc, and combinations thereof. In particular, the thermally conductive material can be fabricated from a thermally conductive metal, thermally conductive metal alloy, thermally conductive composite, or any combination thereof. The thermally conductive metal can be any metal capable of conducting thermal heat such as copper, aluminum, brass, steel, bronze, and other similar materials known to those of skill in the art. The thermally conductive metal alloy can be any combination of metals, chemicals, fillers, and elements containing any percent combination of the constituent components. For example, the thermally conductive metal alloy can be made from combinations of copper, aluminum, brass, steel, bronze, and other similar materials known to those of skill in the art, and include carbon, silica, chemical additives, or elements such as lithium, sodium, or calcium. The thermally conductive composite can be made from any non-metal base material used to conduct heat. For example, carbon material can be compounded in a metal matrix powder of aluminum or the like to fabricate a thermally conductive composite material. In other examples, the composite can be a conductive molded composition made from a polymer base matrix and the like. Other suitable composites known to those of skill in the art are contemplated.

The conductive plate 103 can be sized to be substantially cover an entire side of the layer of silicone rubber 104. In this manner, the heat radiating from the uniformly embedded heating elements can, in turn, be transmitted uniformly through the thermally conductive material of the conductive plate 103. The combination of uniform embedded heating elements and conductive plate can be sized to match the silicone rubber layer to encourage the uniform heating of the item to be heated. The heating elements can heat the silicone rubber 104, which in turn heats the conductive plate 103 and the fluid bag 102. A programmable controller (not shown) in communication with a temperature sensor (not shown) can be programmed to control the electrical current to the heating elements to control heating of the fluid in the fluid bag 102 to a desired temperature. Optionally, the fluid bag 102 can include a handle 105 to assist in transporting the fluid bag 102. In certain embodiments, the heater can have a single heating element embedded, molded, or drowned in the layer of silicone rubber 104. The single heating element can be a serpentine shape, distributed throughout the layer of silicone rubber 104. Alternatively, two or more separate serpentine heating elements can be drowned in the layer of silicone rubber 104. Using two heating elements as a double silicone rubber heater can allow driving half power to each element, and may better control heating. Any number of heating elements can be included in the silicone rubber heater. In certain embodiments, the heating elements can be placed in the same plane, without overlapping of the heating elements in the silicone rubber 104 to provide better heating control. The heating elements can have a lower power density relative to ceramic heaters. Ceramic heaters generally have high heating power densities and smaller dimensions. The silicone rubber heater illustrated in FIG. 1 has a lower heating power density. A lower heating power density can improve temperature control. Because of the high density of a ceramic heater, the temperature of the fluid measured by a temperature sensor depends on the location of the sensor within the bag because the fluid heats unevenly, giving inaccurate readings. With the lower heating power density of the heating elements of the present invention, fluid within a fluid bag such as a peritoneal dialysis fluid bag, can be heated more uniformly, and provide more accurate temperature measurement and control.

As illustrated in FIG. 1B, the dialysis fluid bag 102 can rest on a first side of the conductive plate 103. The first side can be a topside of the conductive plate 103. Once placed on topside of a substantially flat surface, the peritoneal dialysis fluid bag 102 can be heated uniformly by the silicone rubber 104 wherein heat radiates through the conductive plate 103 and into the dialysis fluid bag 102. As illustrated in FIG. 1C, the entirety of the fluid bag 102 can be positioned on a topside of conductive plate 103 and silicone rubber 104. An area of the conductive plate 103 can be substantially similar to an area of the first side of the first layer of silicone rubber 104. As illustrated in FIG. 1D, the fluid bag 102 can be placed on top of the conductive plate 103 and silicone rubber 104 at the top of the peritoneal dialysis system 101. However, other arrangements are contemplated. As illustrated in FIGS. 1A-D, the silicone rubber 104 and conductive plate 103 can be substantially flat. However, because silicone rubber is flexible, in certain embodiments, the silicone rubber 104 and conductive plate 103 can be curved. For example, the silicone rubber 104 and conductive plate 103 can have a concave shape, with the fluid bag 102 fitting into the curve of the concave heater. Thus shape, the heater can be sized to match any surface, cavity, compartment, niche, or section of a device.

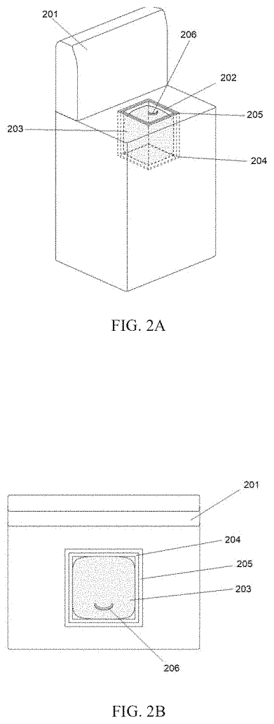

FIGS. 2A-D illustrate a peritoneal dialysis system 201 with a silicone rubber heater lining a receiving compartment 202. FIG. 2A is a perspective view of the peritoneal dialysis system 201, FIG. 2B is a top view of the peritoneal dialysis system 201, FIG. 2C is a side view of peritoneal dialysis system 201, and FIG. 2D shows a fluid bag 203 being placed into the receiving compartment 202. As illustrated in FIG. 2A, the peritoneal dialysis system can include the receiving compartment 202 into which the fluid bag 203 is placed for heating. The receiving compartment in FIGS. 2A-D can be rectangular; however, any shape can be used for the receiving compartment 202. The heater can include a first layer of silicone rubber 205 and a conductive plate 204 positioned on top of the silicone rubber 205. The layer of silicone rubber 205 has embedded heating elements to heat the silicone rubber 205, which in turn heats the conductive plate 204. The fluid bag 203 in contact with the conductive plate 204 is thus heated. Optionally, the fluid bag 203 can include a handle 206 to assist a user transport the fluid bag 203. The receiving compartment 202 can be lined with the silicone rubber 205 and conductive plate 204, whereby the entire receiving compartment 202 can be used to heat the fluid bag 203 placed inside the receiving compartment 202. In certain embodiments, the silicone rubber 205 and conductive plate 204 may not line the entire receiving compartment 202, only lining a bottom portion receiving compartment 202.

As illustrated in FIG. 2B, the fluid bag 203 can fit entirely inside the receiving compartment 202 of the peritoneal dialysis system 201. Once positioned inside the receiving compartment 202, the fluid bag 203 can be heated by the silicone rubber 205 and conductive plate 204. As illustrated in FIG. 2C, the fluid bag 203 can be placed fully within the receiving compartment 202. The silicone rubber 205 and conductive plate 204 can line the inner surface of the receiving compartment 202, contacting the fluid bag 203. As illustrated in FIG. 2D, the fluid bag 203 can be loaded into the receiving compartment 202 from the top of the peritoneal dialysis system 201. However, other arrangements are contemplated. For example, the receiving compartment 202 can be positioned in the front of the peritoneal dialysis system 201 and the fluid bag 203 can be loaded in from the front rather than the top. As described, handle 206 can be included on the fluid bag 203 for easier movement and loading into the receiving compartment 202.

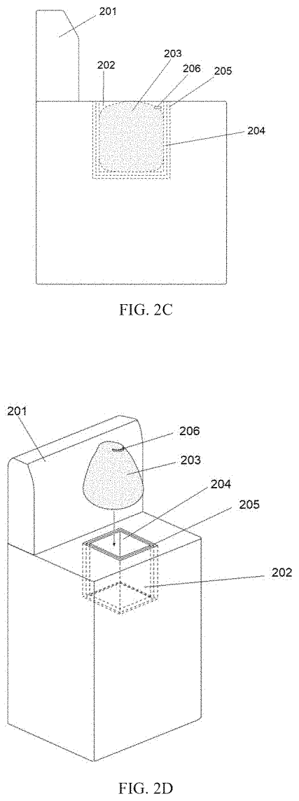

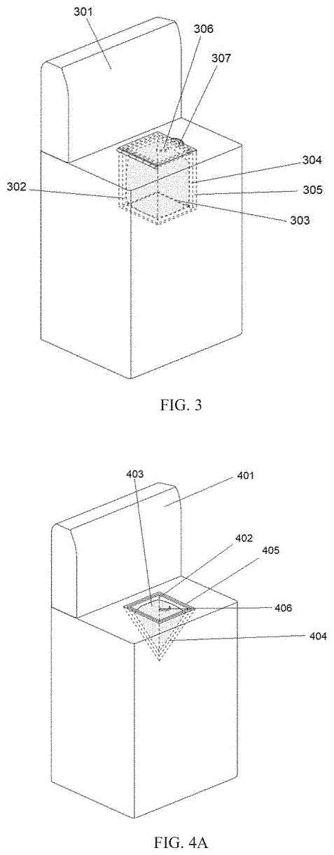

FIG. 3 illustrates a peritoneal dialysis system 301 having a rectangular receiving compartment 302, similar to that shown in FIGS. 2A-D. A layer of silicone rubber 305 and a conductive plate 304 can line all or part of an inner surface of receiving compartment 302 to heat fluid in fluid bag 303. As described, the fluid bag 303 can include handle 306 for easier movement. The embodiment shown in FIG. 3 includes a lid 307 covering the receiving compartment 302. The user can open lid 307, load the fluid bag 303 into the receiving compartment 302, and then close lid 307 to use the heater in a closed system to avoid heat loss and improved efficiency. The lid 307 can also be lined with a layer of silicone rubber and conductive plate.

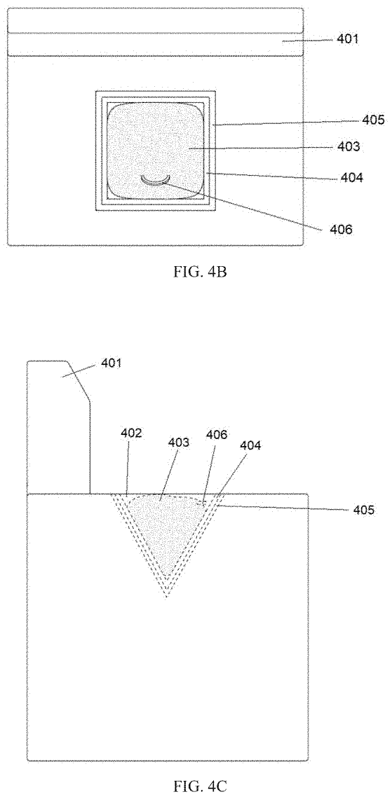

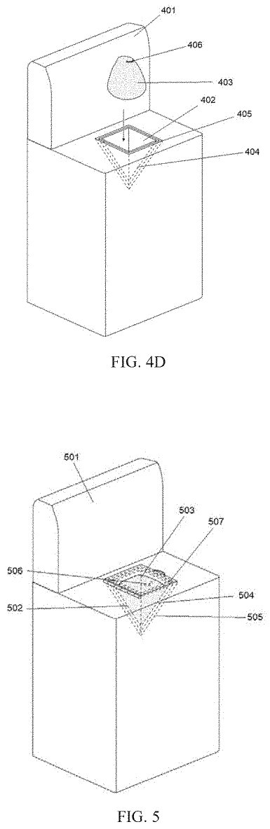

FIGS. 4A-D illustrate a peritoneal dialysis system 401 using a silicone rubber and conductive plate heater in a triangular receiving compartment 402. FIG. 4A is a perspective view of the peritoneal dialysis system 401, FIG. 4B is a top view of the peritoneal dialysis system 401, FIG. 4C is a side view of peritoneal dialysis system 401, and FIG. 4D shows a fluid bag 403 being placed into the triangular receiving compartment 402. As illustrated in FIG. 4A, the peritoneal dialysis system can include a triangular receiving compartment 402 into which the fluid bag 303 can be placed for heating. The heater illustrated in FIGS. 4A-D is similar to that of FIGS. 2A-D but with a triangular rather than rectangular receiving compartment. The heater can include a first layer of silicone rubber 405 and a conductive plate 404 positioned on top of the silicone rubber 405. The layer of silicone rubber 405 has embedded heating elements (not shown) that heat up in response to an electrical current to heat fluid in the fluid bag 403. As described, the fluid bag 403 can include a handle 406 for easier moving of the fluid bag 403.

As illustrated in FIG. 4B, the dialysis fluid bag 403 can fit entirely within the peritoneal dialysis system 401. Once placed inside, the fluid bag 403 can be heated by the silicone rubber 405 and conductive plate 404. As illustrated in FIG. 4C, the fluid bag 403 can be placed fully within the receiving compartment 402. The silicone rubber 405 and conductive plate 404 can line all or part of the inner surface of the receiving compartment 402, contacting the fluid bag 403. As illustrated in FIG. 4D, the fluid bag 403 can be loaded into the receiving compartment 402 from the top of the peritoneal dialysis system 401. However, as described, any other arrangement can be used for the receiving compartment 402.

FIG. 5 illustrates a peritoneal dialysis system 501 having a triangular receiving compartment 502, similar to that shown in FIGS. 4A-D. A layer of silicone rubber 505 and a conductive plate 504 can line the inner surface of receiving compartment 502 to heat fluid in fluid bag 503. As described, the fluid bag 503 can include handle 506 for easier movement. The embodiment shown in FIG. 5 includes a lid 507 covering the receiving compartment 502 allowing the option to enclose the fluid bag 503 in the receiving compartment for heating. The lid 507 can also be lined with a layer of silicone rubber and conductive plate.

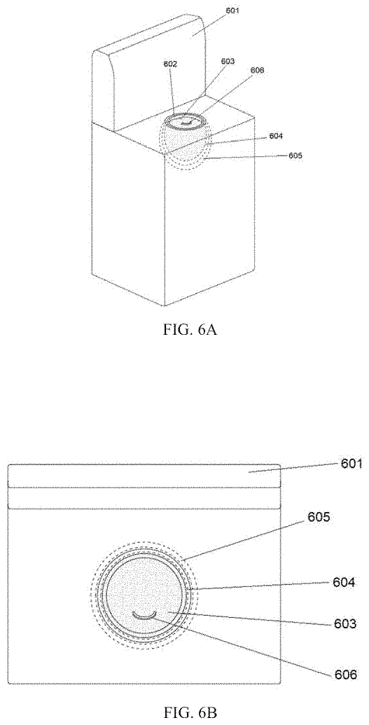

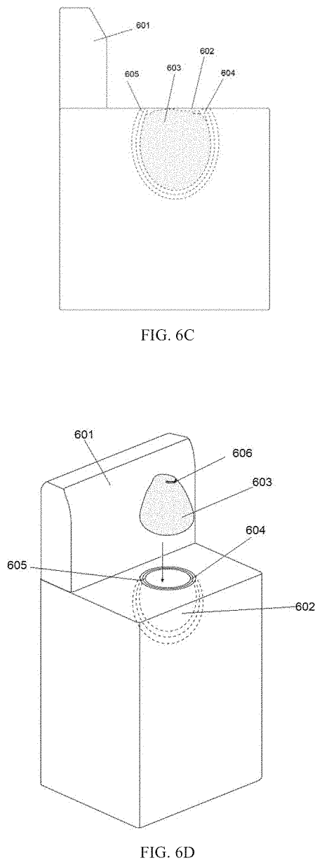

FIGS. 6A-D illustrate a peritoneal dialysis system 601 using a silicone rubber heater in a substantially round receiving compartment 602. FIG. 6A is a perspective view of the peritoneal dialysis system 601, FIG. 6B is a top view of the peritoneal dialysis system 601, FIG. 6C is a side view of peritoneal dialysis system 601, and FIG. 6D shows a fluid bag 603 being placed into the heater. As illustrated in FIG. 6A, the peritoneal dialysis system 601 can include a round receiving compartment 602 into which the fluid bag 603 can be placed for heating. The heater illustrated in FIGS. 6A-D is similar to that of FIGS. 2A-D but with a round rather than rectangular receiving compartment 602. The heater can include a first layer of silicone rubber 605 and a conductive plate 604 positioned on top of the silicone rubber 604. The layer of silicone rubber 605 has embedded heating elements (not shown) that heat up in response to an electrical current to heat fluid in the fluid bag 603. As described, the fluid bag 603 can include a handle 606 for easier moving of the fluid bag 603.

As illustrated in FIG. 6B, the dialysis fluid bag 603 can fit entirely within the peritoneal dialysis system 601. Once placed inside, the fluid bag 603 can be heated by the silicone rubber 605 and conductive plate 604. As illustrated in FIG. 6C, the fluid bag 603 can be placed fully within the receiving compartment 602. The silicone rubber 605 and conductive plate 604 can line the inner surface of the receiving compartment 602, contacting the fluid bag 603. As illustrated in FIG. 6D, the fluid bag 603 can be loaded into the receiving compartment 602 from the top of the peritoneal dialysis system 601. However, as described, any other arrangement can be used for the receiving compartment 602.

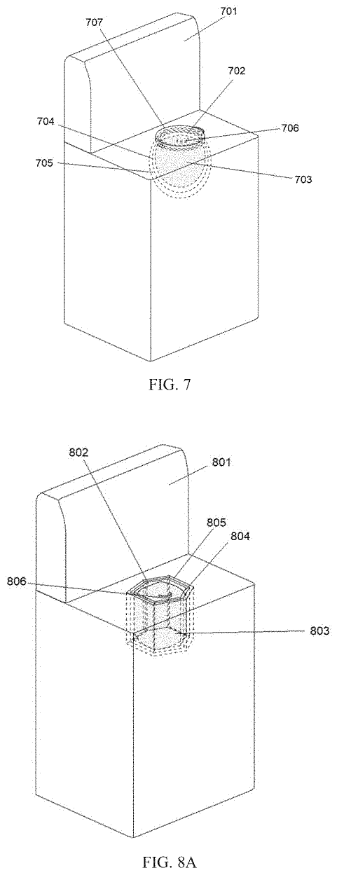

FIG. 7 illustrates a peritoneal dialysis system 701 having a substantially round receiving compartment 702, similar to that shown in FIGS. 6A-D. A layer of silicone rubber 705 and a conductive plate 704 can line the inner surface of receiving compartment 702 to heat fluid in fluid bag 703. As described, the fluid bag 703 can include handle 706 for easier movement. The embodiment shown in FIG. 7 includes a lid 707 covering the receiving compartment 702 allowing the option to enclose the fluid bag 703 in the receiving compartment for heating. The lid 707 can also be lined with a layer of silicone rubber and conductive plate.

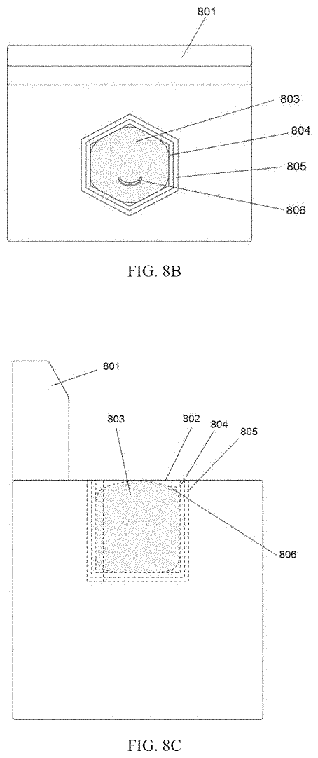

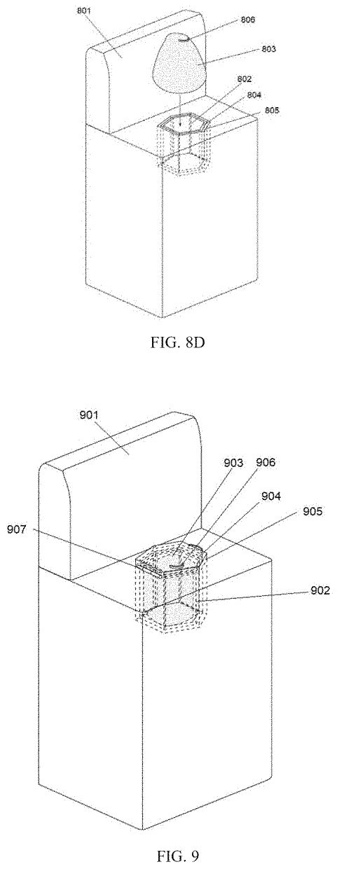

FIGS. 8A-D illustrate a peritoneal dialysis system 801 using a silicone rubber heater in a hexagonal receiving compartment 802. Although shown as hexagonal receiving compartment 802 in FIGS. 8A-D, one of skill in the art will understand that any shape can be used for a receiving compartment. FIG. 8A is a perspective view of the peritoneal dialysis system 801, FIG. 8B is a top view of the peritoneal dialysis system 801, FIG. 8C is a side view of peritoneal dialysis system 801, and FIG. 8D shows a fluid bag 803 being placed into the heater. As illustrated in FIG. 8A, the peritoneal dialysis system can include any shape receiving compartment, such as hexagonal receiving compartment 802 into which the fluid bag 803 can be placed for heating. The heater can include a first layer of silicone rubber 805 and a conductive plate 804 positioned on top of the silicone rubber 804. The layer of silicone rubber 805 has embedded heating elements (not shown) that heat up in response to an electrical current to heat fluid in the fluid bag 803. As described, the fluid bag 803 can include a handle 806 for easier moving of the fluid bag 803.

As illustrated in FIG. 8B, the dialysis fluid bag 803 can fit entirely within the peritoneal dialysis system 801. Once placed inside, the fluid bag 803 can be heated by the silicone rubber 805 and conductive plate 804. As illustrated in FIG. 8C, the fluid bag 803 can be placed fully within the receiving compartment 802. The silicone rubber 805 and conductive plate 804 can line the inner surface of the receiving compartment 802, contacting the fluid bag 803. As illustrated in FIG. 8D, the fluid bag 803 can be loaded into the receiving compartment 802 from the top of the peritoneal dialysis system 801. However, as described, any other arrangement can be used for the receiving compartment 802.

FIG. 9 illustrates a peritoneal dialysis system 901 having a hexagonal receiving compartment 902, similar to that shown in FIGS. 8A-D. A layer of silicone rubber 905 and a conductive plate 904 can line the inner surface of receiving compartment 902 to heat fluid in fluid bag 903. As described, the fluid bag 903 can include handle 906 for easier movement. The embodiment shown in FIG. 9 includes a lid 907 covering the receiving compartment 902 allowing the option to enclose the fluid bag 903 in the receiving compartment for heating. The lid 907 can also be lined with a layer of silicone rubber and conductive plate.

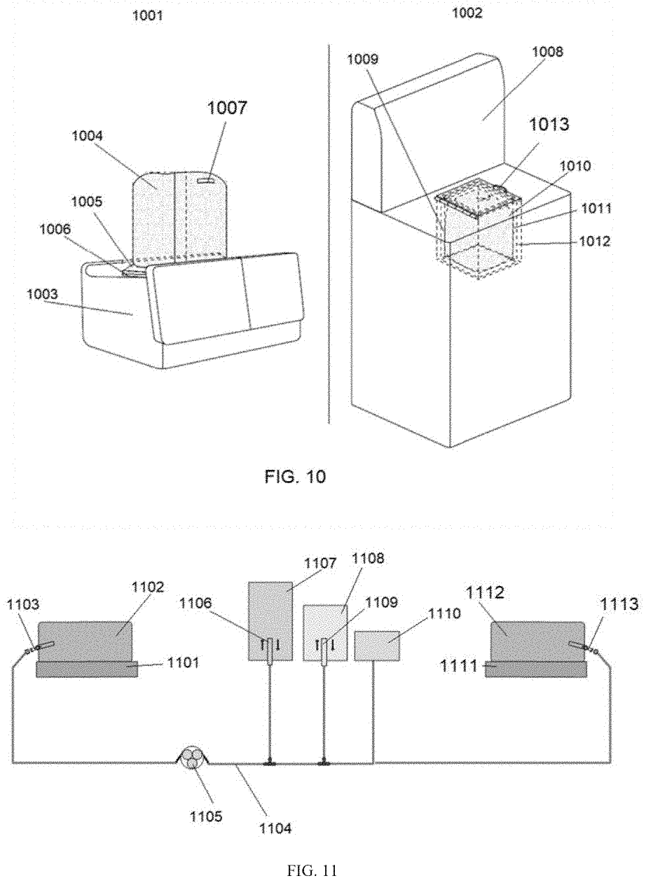

FIG. 10 illustrates a peritoneal dialysis system using two heaters of the invention. The system can include a peritoneal dialysis fluid generation module 1008 and a peritoneal dialysis cycler 1003. The peritoneal dialysis fluid generation module 1008 is used to generate peritoneal dialysis fluid from solid or concentrated components. The peritoneal dialysis cycler 1003 is used to infuse peritoneal dialysis fluid into a patient, and optionally to drain used peritoneal dialysis fluid from the patient. To generate peritoneal dialysis fluid from solid material, the solid material must first be dissolved. A first heater can be used to accurately and precisely heat a source water to a desired temperature to dissolve a solid material, speeding dissolution. The heater can be quickly adjusted to a second temperatures to dissolve a second solid material having different dissolution properties than the first material. As illustrated in FIG. 10, the peritoneal dialysis fluid generation module 1008 can include a receiving compartment 1009 into which a fluid bag 1010 can be placed. The fluid bag 1010 can contain water to be used in dissolving solid components of the peritoneal dialysis fluid. The heater can include a first layer of silicone rubber 1012 with embedded heating elements. A conductive plate 1011 can be positioned on top of the silicone rubber 1012. As described, the heater can heat the fluid in fluid bag 1010 to a desired temperature. The heated fluid can then be pumped into one or more concentrate sources to dissolve the material, which can then be mixed and diluted to generate peritoneal dialysis fluid. The receiving compartment 1009 can optionally include a lid 1013, as described.

Concentrates can be mixed and added into a second fluid bag 1004. In certain embodiments, the concentrates can be individually added to fluid bag 1004 and mixed within the fluid bag 1004. Alternatively, the concentrates can be added to a separate mixing bag or container and the final peritoneal dialysis fluid added to fluid bag 1004. Each concentrate can have a different dissolution profile requiring a different fluid or water temperature. The heater of the invention can quickly and accurately and/or precisely heat the fluid or water to the desired temperature for the specific concentrate.

As illustrated in FIG. 10, the fluid bag 1004 can be placed on top of a silicone rubber heater on a peritoneal dialysis cycler 1003. The heater can include silicone rubber 1006 with embedded heating elements (not shown) and conductive plate 1005 positioned on top of the silicone rubber 1006. The peritoneal dialysis fluid in fluid bag 1004 can be heated to a desired temperature and then infused into the peritoneal cavity of the patient through any means known in the art. In certain embodiments, fluid bag 1004 can include handle 1007 for ease of movement.

As illustrated in FIG. 10, the system can include a separate peritoneal dialysis fluid generation module 1008 and peritoneal dialysis cycler 1003. Two separate heaters can be employed, with one heater for each of the peritoneal dialysis fluid generation module 1008 and peritoneal dialysis cycler 1003. Alternatively, a single module that combines the peritoneal dialysis fluid generation module 1008 and peritoneal dialysis cycler 1003 can be used with a single or multiple heater. Using a separate peritoneal dialysis fluid generation module 1008 and peritoneal dialysis cycler 1003 can enable the components to be spaced apart. For example, the peritoneal dialysis fluid generation module 1008 can be placed in a first room 1002 near to a water supply (not shown). The peritoneal dialysis cycler 1003 can be placed in a separate room 1001 where treatment is more convenient. The user can move the fluid bag 1004 containing the final peritoneal dialysis fluid from the peritoneal dialysis fluid generation module 1008 to the peritoneal dialysis cycler 1003. Alternatively, a tube or hose can carry the peritoneal dialysis fluid into the fluid bag 1004 where the fluid is heated prior to treatment.

Although the heaters illustrated in FIG. 10 are shown as being in a receiving compartment 1009 on the peritoneal dialysis fluid generation module 1008 and on a substantially flat surface on the peritoneal dialysis cycler 1003, one of skill in the art will understand that any arrangement can be used for either module. For example, both modules can use receiving compartments, both modules can use substantially flat surfaces, or any combination can be used.

FIG. 11 illustrates the use of a silicone rubber heater 1101 in a peritoneal dialysis fluid generation module. A fluid bag 1102, which can contain purified water, can be placed on the silicone rubber heater 1101. A controller (not shown) can control the silicone rubber heater 1101 to heat the water in fluid bag 1102 to a desired temperature. Pump 1105 can pump the heated water through an outlet 1103 of fluid bag 1102 into fluid line 1104. The system can include valves and/or additional pumps (not shown) to control the movement of fluid. Purified water can be pumped into a first concentrate source 1107 through connector 1106, into second concentrate source 1108 through connector 1109, and into a third concentrate source 1110. Solid material initially contained in each concentrate source can be dissolved with the water. The concentrates thus generated can then be pumped through fluid line 1104 into a second fluid bag 1112 through inlet 1113 to generate the final peritoneal dialysis fluid. Silicone rubber heater 1111 can heat the final peritoneal dialysis fluid prior to infusion into the patient. As described, in certain embodiments silicone rubber heater 1111 can be part of a separate peritoneal dialysis cycler and need not be positioned as part of the peritoneal dialysis fluid generation module.

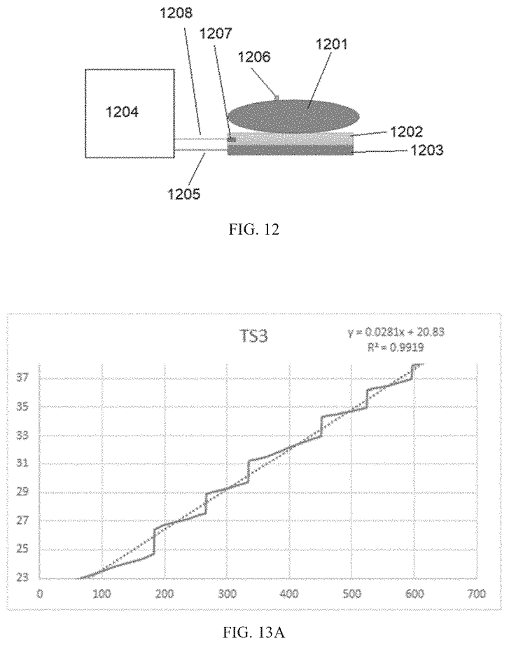

FIG. 12 illustrates an experimental setup to test the silicone rubber heater. An EZ-zone Watlow® heater controller 1204 was used to control heating elements embedded in a layer of silicone rubber 1203. The heater controller 1204 was connected to a PT100 temperature sensor 1207 in conductive plate 1202 by wire 1208. A fluid bag 1201 was placed on top of the conductive plate 1202. A second PT100 temperature sensor 1206 was connected to the fluid bag 1201 to measure the temperature of the fluid. 3 L of peritoneal dialysis fluid at 25° C. was placed inside fluid bag 1201. The temperature of the silicone rubber was set to 60° C. A timer was started and stopped when the temperature of the peritoneal dialysis fluid reached 37° C. The process was repeated with the silicone rubber heater set at each of 65° C. and 70° C. The experiment gives a qualitative determination about the effectiveness of the silicone rubber heater, and in particular, whether the silicone rubber heater can heat 3 L of peritoneal dialysis fluid to 37° C.±2° C.

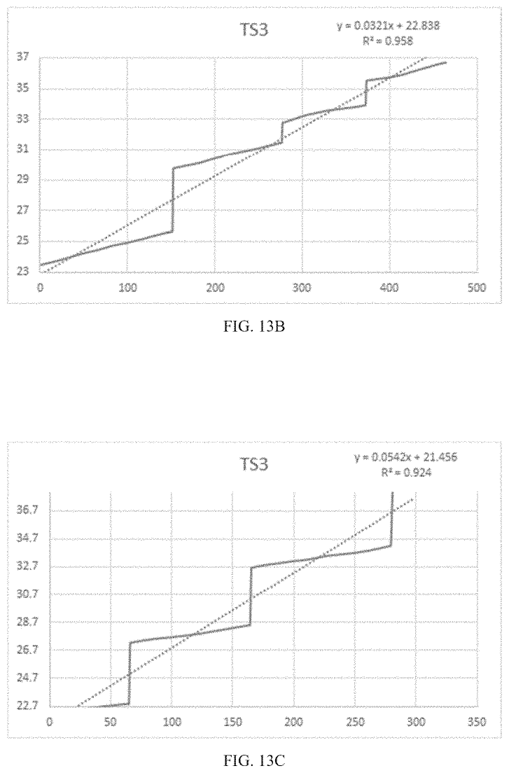

FIGS. 13A-C are graphs of temperature vs. time for the peritoneal dialysis fluid tested with the experimental setup illustrated in FIG. 12. As illustrated in FIG. 13A, with the heater set at 60° C., the peritoneal dialysis fluid reached 37° C. after 527 seconds, or 8.78 minutes. When the heater was set to 65° C., the peritoneal dialysis fluid reached 37° C. after 503 seconds, or 8.38 minutes, as shown in FIG. 13B. With the heater set at 70° C., the peritoneal dialysis fluid reached 37° C. after 519 seconds, or 8.65 minutes, as shown in FIG. 13C. The solid lines in FIGS. 13A-C are the real temperature values plotted by a temperature logger at discrete intervals. The dashed lines in each of FIGS. 13A-C are theoretical trends of the temperature increases Table 1 provides the results for each experiment.

TABLE 1

| | Change in | | |
|---|---|---|---|
| Set Temperature | Temperature (ΔT) | Time | α |
| 60° C. | 13.987° C. | 8.78 min | 1.592° C./min |
| 65° C. | 13.506° C. | 8.38 min | 1.611° C./min |
| 70° C. | 14.154° C. | 8.65 min | 1.636° C./min |

For each set temperature, Table 1 provides the total change in temperature of the peritoneal dialysis fluid, the time to reach 37° C., and α, which is the rate of change in temperature given by ΔT divided by time. Eq(1) provides the value α, where ΔT is the change in temperature and Δt is the change in time. The ΔT varies slightly between the three experiments due to slight variations in starting temperature of the peritoneal dialysis fluid.

$$α = \frac{Δ T}{Δ t} = \frac{{}^{°} C .}{min} \qquad Eq ( 1 )$$

For each set point, the silicone rubber heater was able to heat the peritoneal dialysis fluid effectively to 37° C. The higher the set point used, the quicker the heater is able to heat the peritoneal dialysis fluid, given by value α. In FIGS. 13A-C, a is the angular coefficient of the ideal line, shown as the dashed line for each of FIGS. 13A-C. As shown in Table 1, each setting resulted in heating of the 3 L of peritoneal dialysis fluid at a rate of between 15.9 and 16.4° C./min, illustrating that the silicone rubber heater can effectively heat peritoneal dialysis fluid to a desired temperature quickly with a range of set points used with a starting temperature of between 22-23° C., or room temperature.

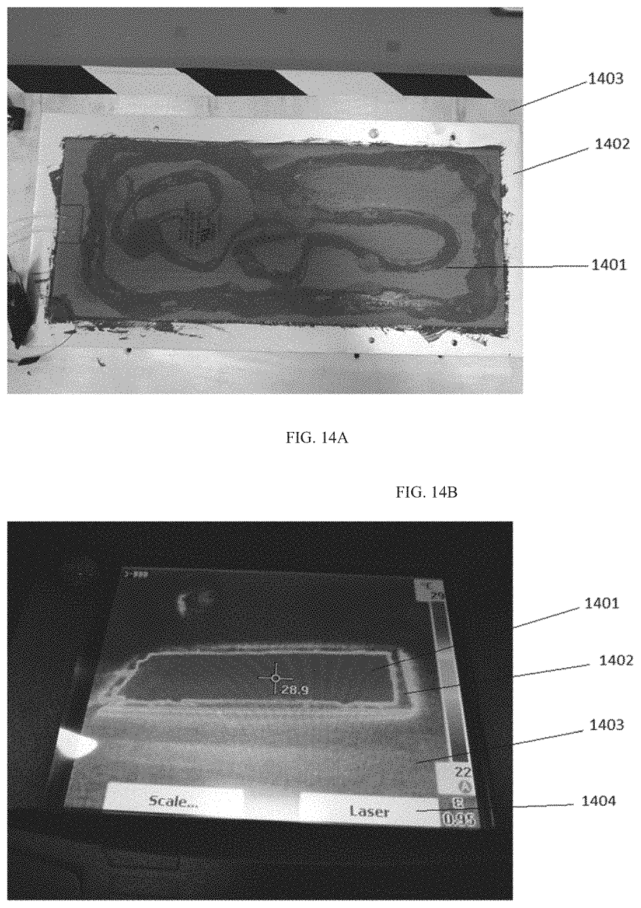

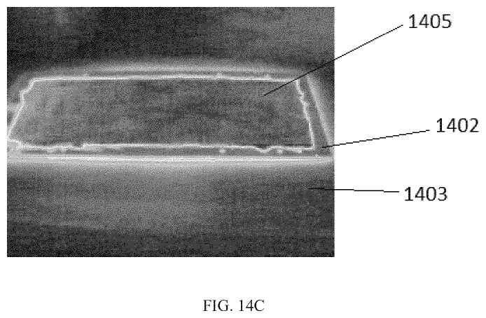

FIGS. 14A-C illustrate an experiment to test the uniformity of heating fluid in a bag using a silicone rubber heater. FIG. 14A is a picture of a deconstructed heater. In FIG. 14A, the heater is shown upside down. A layer of silicone rubber 1401 containing one or more heating elements is shown on top of a conductive plate 1402. The silicone rubber heater was positioned on a wooden plate 1403 as a base to avoid burning the table. In certain embodiments, the conductive plate 1402 and/or layer of silicone rubber 1401 can be attached to the wooden plate 1403. Adhesives or mechanical connectors, such as screws or nails, can be inserted through the conductive plate 1402 and/or layer of silicone rubber 1401 to connect to the wooden plate. The wooden plate can avoid burning of the other components of the dialysis system.

FIG. 14B shows a display screen 1404 with a thermal image of a bag 1405 placed on a silicone rubber heater during heating. The bag 1405 is on top of conductive plate 1402. The heater is placed on a wooden plate 1403. The layer of silicone rubber is not visible in FIG. 14B. As illustrated in FIG. 14B, the bag was a nearly uniform 28.9° C. throughout. The conductive plate 1402 and wooden base 1403 were significantly cooler.

FIG. 14C is a close up of the thermal image illustrated in FIG. 14B. The bag 1405 was nearly uniform in temperature, although slight deviations occurred at the end of the bag 1405. As described, the conductive plate 1402 and wooden base 1403 were significantly cooler. As illustrated in FIGS. 14A-C, the silicone rubber heater described provides effective heating of a fluid bag with a largely uniform temperature distribution.

One skilled in the art will understand that various combinations and/or modifications and variations can be made in the described systems and methods depending upon the specific needs for operation. Various aspects disclosed herein may be combined in different combinations than the combinations specifically presented in the description and accompanying drawings. Moreover, features illustrated or described as being part of an aspect of the disclosure may be used in the aspect of the disclosure, either alone or in combination, or follow a preferred arrangement of one or more of the described elements. Depending on the example, certain acts or events of any of the processes or methods described herein may be performed in a different sequence, may be added, merged, or left out altogether (e.g., certain described acts or events may not be necessary to carry out the techniques). In addition, while certain aspects of this disclosure are described as performed by a single module or unit for purposes of clarity, the techniques of this disclosure may be performed by a combination of units or modules associated with, for example, a medical device.

1. A heater, comprising:
a first layer of silicon rubber (104); the first layer of silicon rubber having one or more heating elements embedded in the silicon rubber; and
a conductive plate (103); the conductive plate positioned on a first side of the first layer of silicon rubber.

2. The heater of claim 1, wherein the conductive plate is selected from any one of an aluminum plate, silver plate, copper plate, gold plate, silicon carbide plate, tungsten plate, graphite plate, zinc plate, and any combination thereof.

3. The heater of claim 1, wherein the conductive plate is selected from a thermally conductive metal alloy or thermally conductive composite.

4. The heater of claim 1, wherein the one or more heating elements are uniformly distributed through the first layer of silicon rubber.

5. The heater of claim 1, wherein the first layer of silicon rubber and the conductive plate have a concave shape.

6. The heater of claim 5, wherein the concave shape is substantially round.

7. The heater of claim 1, wherein an area of the conductive plate is substantially similar to an area of the first side of the first layer of silicon rubber.

8. A system, comprising:
a peritoneal dialysis cycler (1003);
the peritoneal dialysis cycler comprising the heater of claim 1.

9. The system of claim 8, wherein the peritoneal dialysis cycler comprises a substantially flat surface; the first layer of silicon rubber and the conductive plate positioned on the substantially flat surface.

10. The system of claim 8, wherein the peritoneal dialysis cycler comprises a receiving compartment (202) for a fluid bag (203); the first layer of silicon rubber and the conductive plate lining an inner surface of the receiving compartment.

11. The system of claim 10, wherein the receiving compartment is substantially rectangular.

12. The system of claim 10, wherein the receiving compartment is substantially round.

13. A system, comprising:
a peritoneal dialysis fluid generation module (1008); and
the peritoneal dialysis fluid generation module comprising the heater of claim 1.

14. The system of claim 13, wherein the peritoneal fluid generation module comprises a substantially flat surface; the first layer of silicon rubber and the conductive plate positioned on the substantially flat surface.

15. The system of claim 13, wherein the peritoneal dialysis fluid generation module comprises a receiving compartment (1009) for a fluid bag; the first layer of silicon rubber and the conductive plate lining an inner surface of the receiving compartment.

16. The system of claim 15, wherein the receiving compartment is substantially rectangular.

17. The system of claim 15, wherein the receiving compartment is substantially round.

18-24. (canceled)
